(19) United States
(12) Patent Application Publication
(10) Pub. No.: US 2014/0187943 A1
(43) Pub. Date: Jul. 3, 2014

(54) ANGLED NEEDLE ENTRY

(71) Applicant: COOK MEDICAL TECHNOLOGIES LLC, Bloomington, IN (US)

(72) Inventors: Manjiri Dhoke, Bloomington, IN (US); Alina Costin, Bloomington, IN (US); Chris Mobley, Bloomington, IN (US); Lindsay Koren, Bloomington, IN (US); Johan Lowinger, Bloomington, IN (US); Thomas A. Osborne, Bloomington, IN (US)

(73) Assignee: COOK MEDICAL TECHNOLOGIES LLC, Bloomington, IN (US)

(21) Appl. No.: 14/134,374

(22) Filed: Dec. 19, 2013

Related U.S. Application Data
(60) Provisional application No. 61/748,015, filed on Dec. 31, 2012.

(51) Int. Cl. A61B 8/08

(52) U.S. Cl. CPC A61B 8/48
USPC 600/439

(57) ABSTRACT

An angle assist block for transcutaneous insertion of a needle for a patient is provided. The angle assist block comprises a body portion having an outer surface wherein the outer surface comprises a first side and a second side. The first side is configured to engage skin tissue and the second side opposes the first side. The angle assist block further comprises at least one channel formed through the body portion from the first side to the second side. The channel is angled relative to the first side defining an angle and to allow the needle to be inserted through the channel and transcutaneously introduced at the angle. The angle assist block comprises an attachment portion attached to the outer surface. The attachment portion is configured to removably attach to an attachment receiving member of a tissue visualization device.

### CROSS REFERENCE TO RELATED APPLICATIONS

This application claims the benefit of U.S. Provisional Patent Application No. 61/748,015 filed Dec. 31, 2012, the content of which is hereby incorporated by reference in its entirety.

### BACKGROUND

The present invention relates to medical devices. More particularly, the invention relates to an angled assist block to facilitate transcutaneous insertion of a needle for a patient.

Currently, to achieve angled entry, many practitioners rely upon their skill and experience, inserting the needle at the desired angle freehand. With no measurement taken beforehand, this technique is imprecise and does not lead to reproducible punctures. Practitioners may also rely upon a device such as the AXERA Access device to facilitate an angled entry for procedures such as arteriotomy, but these devices require a first puncture step before a second puncture in which angled entry can be performed.

### SUMMARY OF THE INVENTION

In overcoming the drawbacks and other limitations of the related art, embodiments of the present invention provide an angle assist block for transcutaneous insertion of a needle for a patient. In one embodiment, the angle assist block comprises a body portion having an outer surface wherein the outer surface comprises a first side and a second side. The first side is configured to engage skin tissue and the second side opposes the first side. The angle assist block further comprises at least one channel formed through the body portion from the first side to the second side. The channel is angled relative to the first side defining an angle and to allow the needle to be inserted through the channel and transcutaneously introduced at the angle. Moreover, the angle assist block comprises an attachment portion attached to the outer surface. The attachment portion is configured to removably attach to an attachment receiving member of a tissue visualization device.

Further features and advantages of the present disclosure will become apparent from consideration of the following description and the appended claims when taken in connection with the accompanying drawings. It should be understood that the description and specific examples are intended for purposes of illustration only and are not intended to limit the scope of the present disclosure.

### BRIEF DESCRIPTION OF THE DRAWINGS

FIG. 1 is an environmental side view of an angle assist in accordance with one embodiment of the present invention.

FIG. 2 is a cross-sectional view of the angle assist block in FIG. 1 in accordance with the principles of the present invention.

FIG. 3 is a perspective view of an angle assist block with multiple needle entry channels in accordance with another embodiment of the present invention.

FIG. 4 is a cross-sectional view of the angle assist block in FIG. 3 taken along line 4-4.

FIG. 5 is an environmental side view of an angle assist block in accordance with another embodiment of the present invention.

FIG. 6 is an environmental side view of an angle assist block in accordance with yet another embodiment of the present invention.

The drawings described herein are for illustration purposes only and are not intended to limit the scope of the present disclosure in any way.

### DETAILED DESCRIPTION

The present invention generally provides an angle assist device to assist in angled needle entry to transcutaneously access tissues or vessels beneath the skin. Embodiments of the present invention improve the accuracy of angled needle entry by providing a needle with a defined angle of entry and by providing means to attach the angle assist device to a visualization device.

The terms “substantially” or “about” used herein with reference to a quantity includes variations in the recited quantity that are equivalent to the quantity recited, such as an amount that is equivalent to the quantity recited for an intended purpose or function.

One aspect of this invention is to improve a needle entry step of any transcutaneous procedure, such as the Seldinger technique. However, the Seldinger technique is cited here only by way of example and it will be appreciated that all possible techniques that can be envisioned as including an angled entry step is encompassed in the scope of this invention.

An angled needle entry may be the first step in the Seldinger technique. A needle or a trocar is used to puncture the target vessel or cavity. A wire guide is inserted into a lumen of the needle, and the needle is withdrawn and replaced with another operative device, such as a blunt-ended cannula or a drainage tube. The practitioner may then proceed with an endoluminal procedure. When the procedure is complete, the assembly is withdrawn and the puncture wound is closed. Depending on the size of the trocar, the trauma to inner and surface tissues may require extensive sealing after withdrawal. In certain applications of the Seldinger technique, the incision may be dilated, further increasing the size of the area requiring healing and potentially causing excess trauma, such as hemorrhage or perforation, to any layer of the issue or organs affected.

One purpose of angled needle entry is to minimize the amount of bleeding resulting from the Seldinger technique. It does so by maximizing tissue-on-tissue overlap, with the points of piercing of each successive layer of tissue being in contact with intact layers both above (most proximal to the skin) and below (most distal to the skin). This exposes fewer layers directly to the outside environment and promotes self-healing and clotting. It also minimizes the amount of foreign sealing material required to close the wound and decreases the amount of time the wound requires to heal.

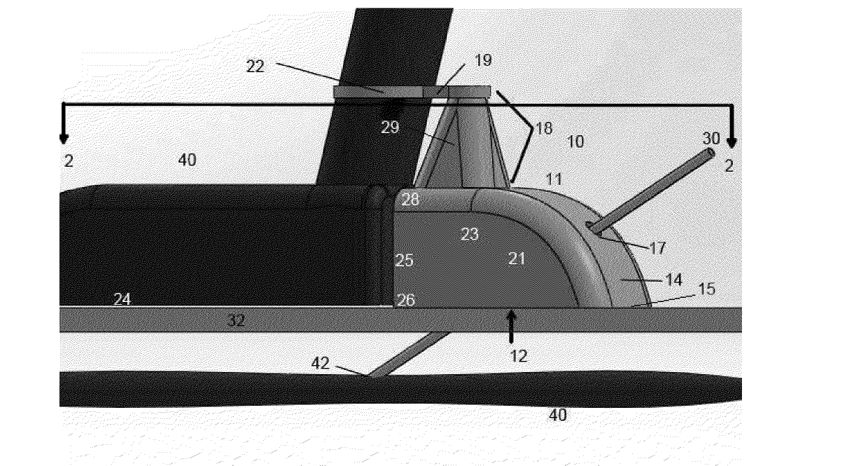

FIG. 1 illustrates an angle assist device 10 in accordance with one embodiment of the present invention. As shown, device 10 may include a visualization device 20 and an angle assist block 11. The angle assist block 11 may work in conjunction with the tissue visualization device 20 to allow the practitioner to view subdermal tissue to precisely target the tissue while at the same time advancing a needle through the tissue at an angle.

The angle assist block 11 may include a body portion 23 and an outer surface 21. The outer surface 21 may include five sides, including a first side 12 that opposes a second side 14. The first side 12 may be configured to engage (e.g. be in contact with) a patient's skin 32. In this embodiment, the first side 12 and the second side 14 may be in contact at edge 15. The outer surface 21 may also include a third side 25 which faces the visualization device 20 and may be positioned directly between the angle assist block 10 and the visualization device 20. The first side 12 and the third side 25 may be in contact at edge 26. The second side 14 and the third side 25 may be in contact at edge 28. The angle assist block 11 may take on other shapes, including those having one or more sides interposed between the first side 12 and the second side 14. The angle assist block 11 may be made of any suitable material, including but not limited to hard plastic or metal that is suitable for interaction with a patient's skin during a medical procedure.

In this embodiment, the first side 12 may have a substantially flat or flat shape, the second side 14 may have a curved shape, and the third side 16 may have a substantially flat or flat shape. However, the first side 12, the second side 14, and the third side 25 may have other shapes. For example, the second side 14 may be orthogonal or substantially orthogonal, parallel or substantially parallel, or at a different angle relative to the first side 12. In some embodiments, both the first side 12 and the second side 14 may be substantially flat or flat and meet at edge 15, and the angle assist block 11 may be in the shape of an five sided block having an inclined plane. In some embodiments, the first side 12 and second side 14 may not meet at edge 15, and may instead be connected by an additional side of the outer surface 21. Moreover, the angle assist block 11 can be of any other shape so long as the block 11 has a first side 12 and second side 14. In some embodiments, the angle assist block 11 may be shaped as a triangular prism.

A channel (e.g. groove) may be formed through and extend through the body portion 23 (e.g. interior) from an entry hole 17 that may be on the first side 12 to an exit hole 27 that may be on the second side 14. The channel may be angled relative to the first side 12 and may define an angle with respect to the first side 12 and/or the skin tissue 32. The needle may be inserted through the channel and penetrate the skin tissue 32 at the angle. The needle may be inserted into entry hole 17, through the channel, out of exit hole 27, and transcutaneously inserted through (e.g. penetrate) the skin 32 at entry point 42 into target tissue 40. The target tissue may be any tissue, vessel, cavity, or organ in which a procedure needs to be performed, for example a vein, an artery, or a chest cavity. In some embodiments, grooves may be formed on the outer surface 21 of the angle assist block 11 rather than through the angle assist block 11. For example, In embodiments where the angle assist block 11 may be a triangular prism, the groove may be cut from or formed on a single side of the outer surface 21 and angled toward the skin 32, and the needle 30 may lie in the groove.

The needle 30 which is shown extending into entry hole 17 can be of any shape or width desired. It will preferably be made of sterilizable metal but can be made of any material that is capable of puncturing skin and subdermal tissue.

Still referring to FIG. 1, the angle assist block 10 may include an attachment portion 18 attached to its outer surface 21. In the embodiment illustrated, the attachment portion 18 may be attached to the second side 14, but the attachment portion may be attached to another portion of the outer surface 21, for example the first side 12 or the third side 25. The attachment portion 18 may have an attachment base 29 and an attachment loop 19. The attachment base 29 may a substantially pyramidal shape that attaches the attachment loop 19 to the body portion 23. The attachment base 29, attachment loop 19, and body portion 23 may be integral with each other, formed of the same material, and/or be a one-piece construction. In other embodiments, the attachment base 29, attachment loop 19, and body portion 23 may each be removably attachable from each other such that the angle assist block 11 can be used independently from the tissue visualization device 20, attachment base 29, and attachment loop 19.

The tissue visualization device 20 may be an ultrasound transducer (e.g. ultrasound probe, ultrasound device). The ultrasound transducer may have a frequency range of 10-5 MHz, for example. However, any other device that allows a practitioner to view the subcutaneous tissue before, during, and after needle entry can be substituted for an ultrasound transducer if desired. Additionally, although the tissue visualization device 20 may, as shown, have an angled L-shape or “hockey stick” shape, any shape of tissue visualization device 20 may be provided and accommodated by the angle assist device 11. The visualization device 20 may be configured to meet the skin 32 at surface 24. The visualization device 20 may include an attachment receiving portion 22 (e.g. a cylindrical or tubular shaft) that may be encircled by the attachment loop 19 of the angle assist block 10. The attachment loop 19 may tightly encircle the attachment receiving portion 22 to prevent relative movement between the attachment loop 19 and the attachment receiving portion 22.

Attachment of the angle assist block 11 to the tissue visualization device may be achieved in a number of ways aide from the embodiment discussed earlier having an attachment loop 19 and/or an attachment base 29. For instance, attachment portion 18 might include of a bolt, while attachment receiving portion 22 of the tissue visualization device may be a bolt-receiving groove. The attachment portion 18 might be an adjustable strap that can be tightened around attachment receiving portion 22. The angle assist block and the visualization devices may be manufactured in such a way that their housings slide together or snap together and can be disconnected after use. For example, the attachment portion 18 may include a tab that lockingly engages a latch of the attachment receiving portion 22 to form a quick-release mount between the angle assist block 11 and the visualization device 20. In another example, the attachment portion 18 may include a surface (e.g. lip) that is configured to lockingly engage a groove of attachment receiving portion 22. The groove may be a part of a shoe-type connector. In some embodiments, a button may be provided to retract a part of the attachment receiving portion 22 (e.g. tab or shoe-type connector) from the attachment portion 18 (e.g. latch or lip). For especially delicate procedures, the angle assist block 11 might contact the visualization device 20 at multiple attachment points or across an extended surface in order to impart better stability to the assembly. Each of the attachment portions 18 may be attached to the third side 25.

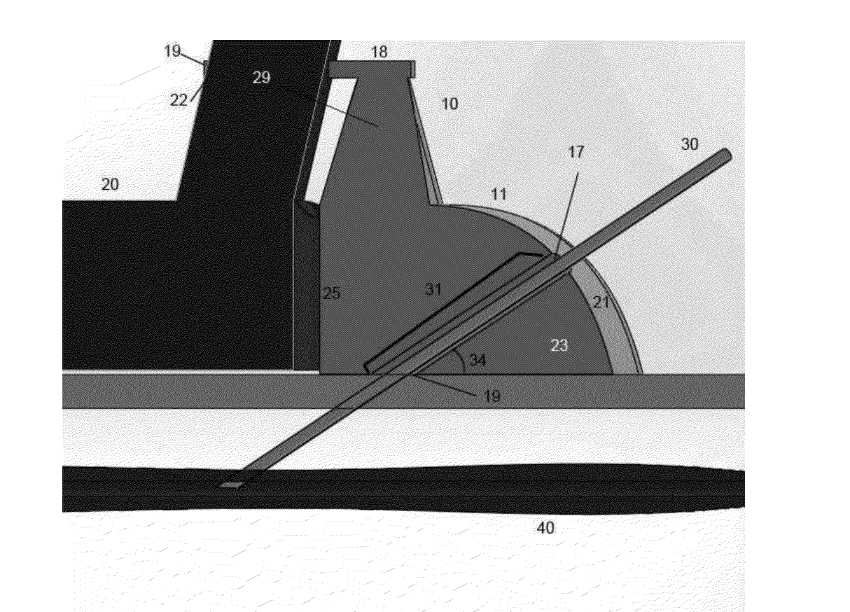

Turning now to FIG. 2, a cross-sectional view of the assembly of FIG. 1 is illustrated. FIG. 2 shows internal features of angle assist block 11, for example the needle channel 31 and needle exit hole 27. Needle exit hole 27 may be formed through the first side 12 of the angle assist block 11 and may be the portion of the device that enables the needle 30 to make contact with and puncture the skin 32. The needle entry channel 31 may be formed during the manufacture of the block itself, such as by allowing shapeable plastic to form around a channel-forming object, or the channel 31 may be created by a method such as drilling or puncturing the block with an appropriate tool after the angle assist block 11 itself has been made.

The needle channel 31 may be angled at an angle 34 relative to the first side 12 and/or the surface of the skin with the skin 32. The needle may enter the body at angle 34. The angle 34 may be suitable angle, for example the angle 34 may depend upon the length of the entry path through the target tissue 40 and all tissue layers between the target tissue 40 and the skin 32, as dictated by the procedure. The angle 34 may be an acute angle, for example below 90 degrees and above 0 degrees, between about 1 degrees and about 89 degrees, between about 10 degrees and about 80 degrees, between about 15 degrees and about 75 degrees, between about 20 degrees and about 70 degrees, between about 25 degrees and 65 degrees, between about 30 degrees and 60 degrees, between about 35 degrees and 55 degrees, between about 40 degrees and 50 degrees, or about 45 degrees.

The needle channel 31, the needle entry hole 17, and the exit hole 27 may be cylindrical in shape, with the needle channel 31 having a smooth internal surface. In other embodiments, the needle channel 31, the needle entry hole 17, and the exit hole 27 may be square or rectangular. The needle channel 31 may also have smaller grooves formed into part or all of its length to provide better guidance for the needle 30. There may be a multitude of other shapes of the holes and further types of striations (e.g. ridges) along the interior of the needle entry channel 31 that still fall within the scope of this invention.

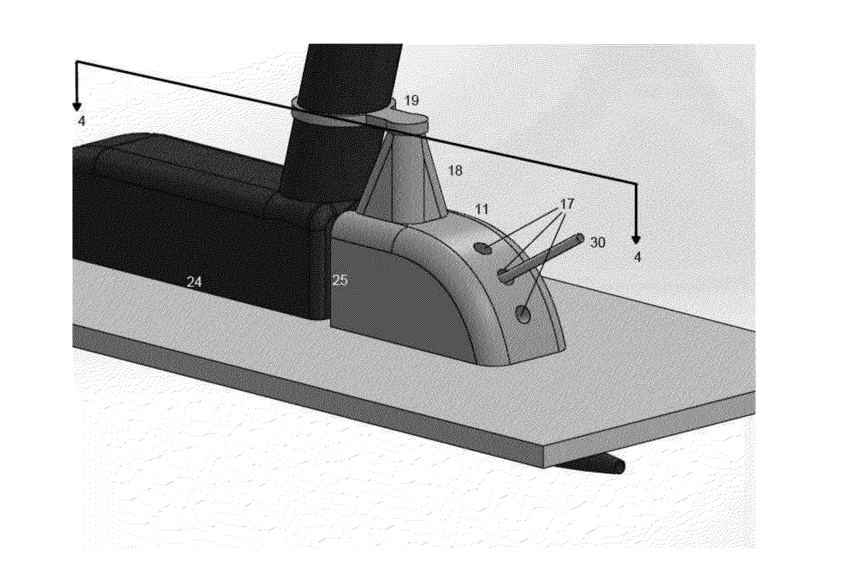

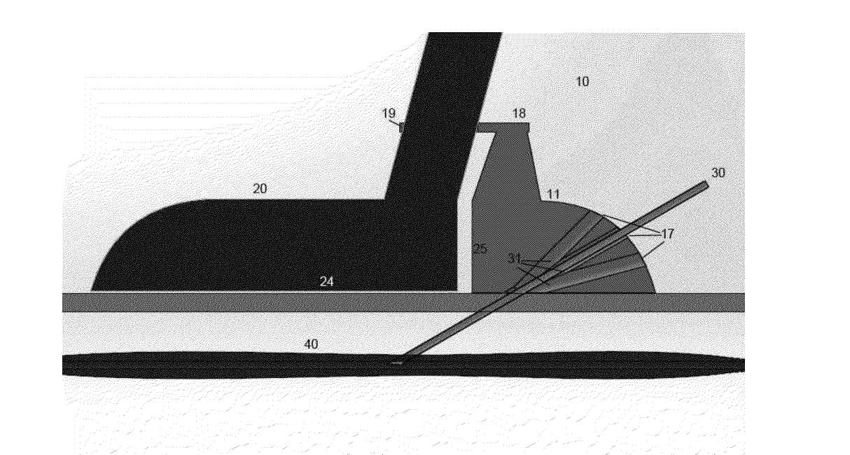

FIGS. 3 and 4 illustrate another embodiment of an angle assist device 10 which may be similar to the angle assist device of FIGS. 1 and 2. However, in this embodiment, angle assist block 10 may have a plurality of needle entry holes 17. As illustrated in the cross-sectional view of FIG. 4, this plurality of entry holes may extend in respective channels which may terminate at a single needle exit hole 27 through the first side 12. In some embodiments, each entry hole 17 may have its own corresponding exit hole 27. Additionally, in some embodiments, the needle entry channels may intersect or may be constructed in a non-intersecting fashion. Thus, a single entry hole 17 may lead to a single or multiple exit holes 27. The needle entry channels may be angled in parallel, substantially parallel, or non-parallel to each other. The entry holes 17 may be spaced apart and disposed on the second side 14 along a longitudinal axis that may extend through the edge 15, the third side 25, and/or the visualization device 20 which may be orthogonal or substantially orthogonal to the edge 15. The longitudinal axis may be coplanar or substantially coplanar with the channels 31. An angle assist block with multiple needle channels may enable a practitioner to use a single piece of equipment for a variety of tasks involving percutaneous procedures. Although three entry holes 17 and three channels are shown, there may be two, four, five, six, seven, eight, nine, ten, or any number of entry holes 17, two, four, five, six, seven, eight, nine, ten, or any number of channels, two, four, five, six, seven, eight, nine, ten, or any number of exit holes 27 .

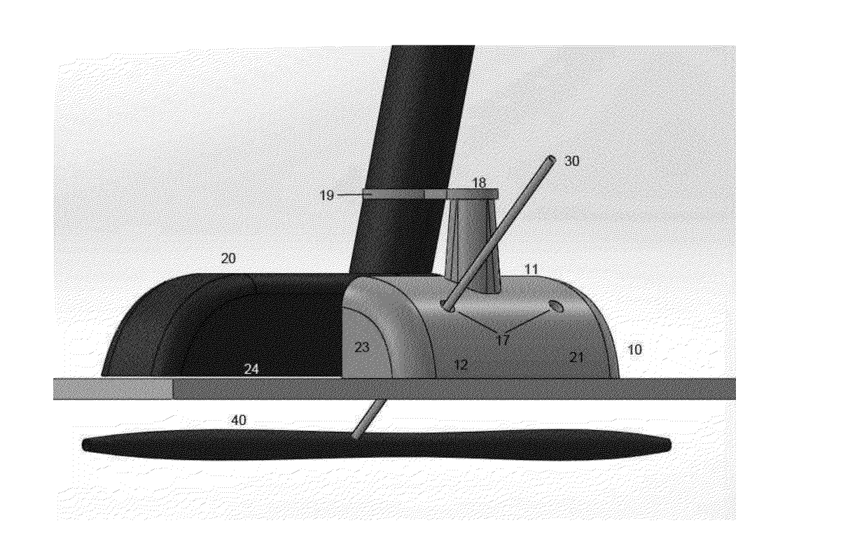

FIG. 5 illustrates yet another embodiment of this invention which may be similar to the embodiment of FIGS. 3 and 4. However, this embodiment may include multiple needle entry holes 17 that may be spaced apart and disposed on the second side 14 along a longitudinal axis that may be parallel or substantially parallel to the edge 15. The longitudinal axis may be coplanar or substantially coplanar with the channels 31. This embodiment may be used when multiple punctures may be made to a target tissue at different points along the length of the target tissue, or when multiple subcutaneous structures may be accessed. Although only two needle entry holes 17 are illustrated here, it will be understood that the number of needle entry holes may be limited only by the size of the angle assist block itself and the diameter of the channels required to fit the needle therein. For example, although two entry holes 17, two channels, and two exit holes 27 are shown, there may be three, four, five, six, seven, eight, nine, ten, or any number of entry holes 17, three, four, five, six, seven, eight, nine, ten, or any number of channels, three, four, five, six, seven, eight, nine, ten, or any number of exit holes 27.

Additionally, although the embodiments of FIGS. 3-5 show entry holes 17 disposed along a single longitudinal axis, the entry holes 17, channels 31, and exit holes 19 may be spaced apart along both longitudinal axes (e.g. two orthogonal longitudinal axes) such that they form a grid of entry holes 17, channels 31 (some or all of which may be parallel or substantially parallel, for example), and exit holes 27. For example, the grid may be a 2×2 grid or 3×3 grid.

FIGS. 1-5 illustrate some possible relative positioning schemes for the angle assist block 11 relative to the tissue visualization device 20. However, other relative positioning schemes may be implemented. For example, an attachment receiving point of the attachment receiving portion 22 may be a single point, or may run down the entire contacting surfaces of both the attachment portion 18 and the attachment receiving portion 22. If the angle assist block 11 or the visualization device 20 have a first set of edges that are narrower than a second set of edges (e.g. where the second set of edges are orthogonal or substantially orthogonal to the first set of edges), or both, then a narrower edge of the angle assist block 11 may face and/or contact a narrow edge of the tissue visualization device 20 as in FIG. 1, or a wider edge of the angle assist block 11 may face and/or contact a wider edge of the tissue visualization device 20 as in FIG. 5. In some embodiments, a wider edge may face and/or contact a narrower edge. There may be a plurality of contacts made between the angle assist block 11 and the tissue visualization device 20, or there may be just a single contact. There may be any number of spaces between the block 11 and the visualization device 20, or the two may be completely integral with each other along the sides at which they meet.

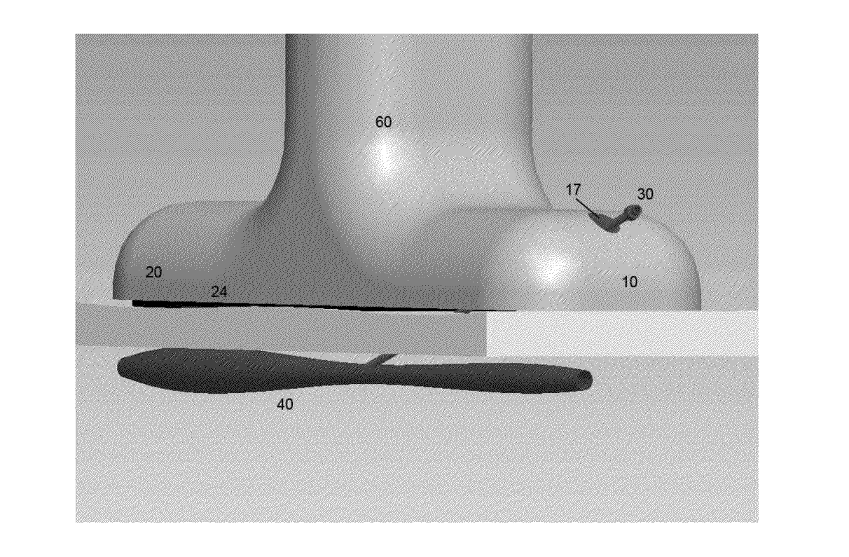

Turning now to FIG. 6, yet another embodiment of the invention is illustrated. This embodiment may be similar to the embodiments of FIG. 1-5. However, in this embodiment, the angle assist block portion 11 and the tissue visualization device 20 may be components assembled within a single housing 60 and may therefore be integral with each other, made of the same material as each other, and/or may be formed of a one-piece construction with each other. Although the housing 60 may be a single housing 60, this embodiment may also encompass any attachment of an irreversible or difficult-to-reverse nature, such as welding, soldering, gluing, and the like.

The outer housing 60 of the device of this embodiment may be made of any suitable material that may not interfere with the operation of the tissue visualization device 20 housed within, including various plastics or metals or a combination thereof. The options for configuring the angle assist block portion of this unified device may encompass the options contemplated in the embodiments of FIG. 1-5. For example, a plurality of needle exit holes 27 and entry holes 17 may be formed through the first side 12 and second side 12, respectively. The channels 31 may be formed at a variety of angles or identical or substantially identical angles, and may intersect or not intersect. The shapes of the holes 17 and 27 and the channels 31, as well as their diameters, lengths, and depths, may be varied as well, in accordance with the needles or trocars intended to be used in the application for which the device is designed.

As a person skilled in the art will readily appreciate, the above description is meant as an illustration of implementation of the principles of this invention. This description is not intended to limit the scope or application of this invention in that the invention is susceptible to modification, variation and change, without departing from the spirit of this invention, as defined in the following claims.

1. An angle assist block for transcutaneous insertion of a needle for a patient, the angle assist block comprising:
a body portion having an outer surface, the outer surface comprising a first side and a second side, the first side being configured to engage skin tissue, the second side opposing the first side;
at least one channel formed through the body portion from the first side to the second side, the channel being angled relative to the first side defining an angle and to allow the needle to be inserted through the channel and transcutaneously introduced through the skin tissue at the angle; and
an attachment portion attached to the outer surface, the attachment portion being configured to removably attach to an attachment receiving portion of a tissue visualization device.

2. The angle assist block of claim 1 wherein the tissue visualization device comprises an ultrasound transducer.

3. The angle assist block of claim 1 wherein the attachment portion comprises a clamp, and wherein the attachment receiving portion comprises a clamp-receiving portion.

4. The angle assist block of claim 1 wherein the attachment portion comprises a bolt, and wherein the attachment receiving portion comprises a bolt-receiving portion.

5. The angle assist block of claim 1 wherein the attachment portion comprises a strap, and wherein the attachment receiving portion comprises a strap-receiving portion.

6. The angle assist block of claim 1 wherein the attachment portion comprises a receiving loop having an inner diameter through which the attachment receiving portion of the visualization device extends.

7. The angle assist block of claim 1 wherein the at least one channel comprises a plurality of channels.

8. The angle assist block of claim 7 wherein the angle of a first channel of the plurality of channels is substantially identical to an angle of a second channel of the plurality of channels.

9. The angle assist device of claim 7 wherein the angle of a first channel of the plurality of channels is different relative an angle of a second channel of the plurality of channels.

10. The angle assist device of claim 1 wherein the first side and the second side share an edge.

11. The angle assist device of claim 1 wherein the first side and the second side do share an edge.

12. An apparatus comprising:
a tissue visualization device having an attachment receiving portion; and
an angle assist block comprising:
a body portion having an outer surface, the outer surface comprising a first side and a second side, the first side being configured to engage skin tissue, the second side opposing the first side, the body portion being integral with the tissue visualization device;
at least one channel formed through the body portion from the first side to the second side, the channel being angled relative to the first side defining an angle and to allow the needle to be inserted through the channel and transcutaneously introduced penetrate the skin tissue at the angle.

13. The apparatus of claim 12 wherein the tissue visualization device comprises an ultrasound transducer.

14. The apparatus of claim 12 further comprising an exterior housing, wherein the tissue visualization device and the body portion are formed of the exterior housing.

15. The apparatus of claim 12 wherein the at least one channel comprises a plurality of channels.

16. The apparatus of claim 12 wherein the angle of a first channel of the plurality of channels is substantially identical to an angle of a second channel of the plurality of channels.

17. The apparatus of claim 12 wherein the angle of a first channel of the plurality of channels is different relative an angle of a second channel of the plurality of channels.

18. The apparatus of claim 17 wherein the first side and the second side share an edge.

19. The apparatus of claim 17 wherein the first side and the second side do share an edge.

20. A method of using an angle assist block and a visualization device, the angle assist block comprising a body portion having an outer surface, the outer surface comprising a first side and a second side, the first side being configured to engage skin tissue, the second side opposing the first side, the angle assist block further comprising at least one channel formed through the body portion from the first side to the second side, the channel being angled relative to the first side defining an angle, the angle assist block further comprising an attachment portion attached to the outer surface, the attachment portion being configured to removably attach to an attachment receiving portion of the visualization device, the method comprising:
providing a view of internal body tissue with the visualization device;
inserting a needle through the channel; and
while providing the view of the internal body tissue, transcutaneously introducing the needle through the skin tissue at the angle.
